(19) United States
(12) Patent Application Publication
(10) Pub. No.: US 2026/0146075 A1
(43) Pub. Date: May 28, 2026

(54) RECOMBINANT TYPE III HUMAN-DERIVED COLLAGEN PEPTIDE FOR ANTI-HUMAN PAPILLOMAVIRUS (HPV) INFECTION AND METHOD OF PREPARATION AND USE THEREOF

(71) Applicant: Shanxi Academy of Traditional Chinese Medicine, Taiyuan (CN)

(72) Inventors: Chongzhi BAI, Taiyuan (CN); Ruoyu WANG, Taiyuan (CN); Qian YANG, Taiyuan (CN); Liguo TONG, Taiyuan (CN); Haijie JI, Taiyuan (CN); Qiming ZHONG, Taiyuan (CN); Shu WANG, Taiyuan (CN); Meiqing SONG, Taiyuan (CN); Tingyu LI, Taiyuan (CN); Chen ZHENG, Taiyuan (CN); Yan GAO, Taiyuan (CN)

(21) Appl. No.: 19/382,801

(22) Filed: Nov. 7, 2025

(30) Foreign Application Priority Data
Nov. 28, 2024 (CN) 202411722935.0

(51) Int. Cl. C07K 14/78; A61K 38/00; A61P 31/20

(52) U.S. Cl. CPC C07K 14/78; A61P 31/20; A61K 38/00

(57) ABSTRACT

A recombinant type III human-derived collagen peptide for anti-HPV infection, a method preparation, and use thereof are provided. The amino acid sequence of the peptide is derived from a sequence encoding positions 991 to 1032 of type III human-derived collagen (UniProtKB: P02461), repeated three times, and is named ATHPV3, with the amino acid sequence set forth in SEQ ID NO. 1. After expression and purification, a recombinant type III human-derived collagen peptide with a molecular weight of approximately 13 kD is obtained, which can specifically bind to HPV 16/18 L1 proteins and forms multiple hydrogen bonds.

### CROSS-REFERENCE TO RELATED APPLICATION

This patent application claims the benefit and priority of Chinese Patent Application No. 202411722935.0 filed with the China National Intellectual Property Administration on Nov. 28, 2024, the disclosure of which is incorporated by reference herein in its entirety as part of the present application.

### REFERENCE TO SEQUENCE LISTING

A computer readable XML file entitled “2024117229350_seqlist”, which was created on Oct. 24, 2025, with a file size of about 3,598 bytes, contains the sequence listing for this application, has been filed with this application, and is hereby incorporated by reference in its entirety.

### TECHNICAL FIELD

The present disclosure belongs to the field of biomedical technology, and particularly relates to a recombinant type III human-derived collagen peptide for anti-human papillomavirus (HPV) infection, a method of preparation, and use thereof. More specifically, it pertains to a recombinant type III human-derived collagen peptide that binds to the HPV L1 protein and use thereof in the preparation of a medicament for the prevention and/or treatment of HPV infections.

### BACKGROUND

Human papillomavirus (HPV) is a circular DNA virus that predominantly affects the squamous epithelium of the skin and mucous membranes. The infection rate of HPV among healthy women in different regions of China ranges from 15% to 28%. Persistent HPV infection is the main cause of cervical cancer, with 70% to 84% of cervical cancer cases associated with infections from HPV types 16 and 18. Apart from vaccination as a preventive measure, there are currently no specific medications available. Existing treatments mainly rely on physical therapies and adjunctive treatments like interferons, which are insufficient in addressing subclinical and persistent infections. This leads to a situation where symptoms are alleviated but the underlying issue remains, making recurrence likely. For patients with high-risk HPV infections that cannot be self-cleared, it is of great significance to block the persistent infection of the virus, reduce viral load, and thus prevent cervical lesions. Type III human collagen is widely present in human tissues and plays important structural and functional roles, primarily distributed in the skin, blood vessels, muscles, intestinal mucosa, and the outer membranes of organs. Exogenous supplementation of active human type III collagen can provide lubrication and moisturizing effects for the vagina, restore the normal tissue functions of the vaginal mucosa and vaginal elasticity, and help maintain the normal function of the cervix.

### SUMMARY

The present disclosure provides a recombinant type III human-derived collagen peptide for anti-human papillomavirus (HPV) infection, a method of preparation, and use thereof. More specifically, the present disclosure pertains to a recombinant type III human-derived collagen peptide that binds to the HPV L1 protein and use thereof in the preparation of a medicament for the prevention and/or treatment of HPV infections.

The present disclosure is embodied by the following technical solutions. Provided is a recombinant type III human-derived collagen peptide for anti-HPV infection, with the amino acid sequence derived from positions 991 to 1032 of a type III human-derived collagen encoded by UniProtKB: P02461, repeated three times, named ATHPV3, and the specific amino acid sequence is set forth SEQ ID NO: 1.

The present disclosure also provides a gene encoding the recombinant type III human-derived collagen peptide for anti-HPV infection, having the nucleotide sequence of SEQ ID NO. 2.

The present disclosure further provides a method for preparing the recombinant type III human-derived collagen peptide for anti-HPV infection, the method including the following steps:
Cultivation: Triplicating the amino acid sequence from positions 991 to 1032 of a type III human-derived collagen encoded by UniProtKB: P02461 and named ATHPV3; ligating a 3′ end of the amino acid sequence to a histidine tag and a termination codon (TGA), and constructing a resulting sequence into a plasmid Cytomegalovirus (CMV)-Actin Globin Gateway Shuttle (pCAGGS) vector through restriction enzyme sites EcoRI and XhoI, transfecting a resulting vector into a human embryonic kidney (HEK) 293-F cell for eukaryotic expression, and collecting a supernatant after the HEK 293F cell is cultured;
Elution: Purifying a target protein using a HisTrap HP 5 mL affinity chromatography column, and eluting the purified protein with a buffer containing pH 8.0, 20 mM Tris, 150 mM NaCl, and 300 mM imidazole; and
Purification: Purifying the eluted protein using a HiLoad 16/600 Superdex 75 prep gel filtration chromatography column in a buffer containing pH 8.0, 20 mM Tris, and 150 mM NaCl, at a flow rate of 1 ml/min; Identifying the purified protein by sodium dodecyl sulfate-polyacrylamide gel electrophoresis (SDS-PAGE), and determining a protein with a molecular weight of 13 kDa as a target collagen peptide.

The present disclosure also provides use of the recombinant type III human-derived collagen peptide for anti-HPV infection in the preparation of a medicament for the prevention and/or treatment of an HPV infection.

In some embodiments, the recombinant type III human-derived collagen peptide ATHPV3 binds to HPV 16/18 L1 proteins, with affinities of 51.6 nM and 58.2 nM, respectively.

In some embodiments, the recombinant type III human-derived collagen peptide ATHPV3 forms five hydrogen bonds with HPV16 L1 and four hydrogen bonds with HPV18 L1.

The present disclosure is based on a human type III collagen sequence, which has been truncated and modified to design a recombinant protein peptide ATHPV3 that binds to the HPV L1 protein. This peptide is then transfected into a plasmid suitable for expression in HEK 293F cells for the production of the recombinant type III human-derived collagen peptide. The affinity of the recombinant type III human-derived collagen peptide for HPV 16/18 L1 proteins is detected using the surface plasmon resonance (SPR) method; the interaction between the two proteins is proved by molecular docking; the neutralizing activity of the recombinant type III human-derived collagen peptide against HPV 16/18 pseudoviruses is tested by a luciferase reporter gene assay.

The results show that through expression and purification, a recombinant type III human-derived collagen peptide with a molecular weight of approximately 13 kD is obtained. Compared to the reference sequences with eight repeats (about 30 kD) and ten repeats (about 40 kD), the recombinant peptide with smaller molecular weight allows for better penetration into the target protein structural domains. Affinity results indicate that ATHPV3 may effectively bind to HPV 16/18 L1 proteins with high specificity; there are multiple hydrogen bond interactions between ATHPV3 and HPV 16/18 L1 proteins. Both high and medium concentrations of ATHPV3 can inhibit the pseudovirus infection of 293T cells. For the body, the collagen peptide produced by the present disclosure may inhibit HPV invasion into target cells by binding to HPV L1, preventing viral replication, and reducing viral load, thereby blocking persistent viral infection and achieving prevention and treatment of cervical cancer and other diseases caused by HPV.

The ATHPV3 prepared by the present disclosure exhibits significant anti-HPV activity, providing an effective method for treating diseases caused by HPV infections, such as cervical cancer. In the present disclosure, successfully obtained is a recombinant type III human-derived collagen peptide ATHPV3 with good purity, capable of binding to HPV 16/18 L1 and effectively inhibiting HPV 16/18 pseudovirus infection in 293T cells, offering an effective candidate drug for the prevention and treatment of HPV infections.

### BRIEF DESCRIPTION OF THE DRAWINGS

FIG. 1 shows the results of gel electrophoresis of the purified ATHPV3. FIGS. 2A-2B show the SDS-PAGE identification results of the purified ATHPV3, where FIG. 2A shows the purification results of the collagen peptide; FIG. 2B shows the SDS-PAGE electrophoresis results of the collagen peptide. FIGS. 3A-3D show the results of SPR assay detecting the binding affinity of the recombinant type III human-derived collagen peptide to HPV 16/18 L1 proteins, where FIG. 3A shows the binding affinity detection result of the negative control 2F2E5 with HPV16 L1 protein; FIG. 3B shows the binding affinity detection result of the negative control 2F2E5 with HPV18 L1 protein; FIG. 3C shows the binding affinity detection result of the collagen peptide with HPV16 L1 protein; FIG. 3D shows the binding affinity detection result of the collagen peptide with HPV18 L1 protein. FIGS. 4A-4B illustrate the interaction between the collagen peptide and HPV16/18 L1 proteins, where FIG. 4A shows the interaction between the collagen peptide and HPV16 L1; FIG. 4B shows the interaction of the collagen peptide with HPV18 L1. FIG. 5 presents the results of the luciferase reporter gene assay detecting the neutralizing activity of the recombinant type III human-derived collagen peptide against HPV 16/18 pseudoviruses.

### DETAILED DESCRIPTION OF THE EMBODIMENTS

To make the objectives, technical solutions, and advantages of the embodiments of the present invention clearer, the technical solutions in the examples of the present disclosure will be described clearly and completely below. Obviously, the described examples are part of the embodiments of the present disclosure and are not all of them. All other embodiments obtained by those skilled in the art based on the examples of the present disclosure, without making any creative efforts, belong to the protection scope of the present disclosure.

Unless otherwise defined, all technical and scientific terms used herein have the same meanings as commonly understood by those skilled in the art to which the present disclosure pertains. Materials cited herein and those referenced will be incorporated by reference.

Those skilled in the art will recognize that equivalent technologies to the specific embodiments described, which can be understood through routine experimentation, are included in the present disclosure.

The experimental methods in the following examples are all conventional methods unless specifically noted otherwise. The instruments and equipment used in the following examples are all standard laboratory instruments unless specified otherwise. The experimental materials used in the following examples are all commercially available from conventional biochemical reagent suppliers unless specified otherwise.

I. Construction of Recombinant Type III Human-Derived Collagen Peptide: The coding sequence for positions 991 to 1032 of type III human-derived collagen (UniProtKB: P02461) was repeated three times, named ATHPV3, with the specific amino acid sequence set forth in SEQ ID NO. 1, as follows:

GPIGPPGPRGNRGERGSEGSPGHPGMPGPPGPPGAPGPCCGGGPIGPPG
PRGNRGERGSEGSPGHPGMPGPPGPPGAPGPCCGGGPIGPPGPRGNRGE
RGSEGSPGHPGMPGPPGPPGAPGPCCGG.

Then, a histidine tag and a termination codon (TGA) were ligated at the 3′ end, and the sequence was constructed into the pCAGGS vector through the restriction enzyme sites EcoRI and XhoI, which was then transfected into HEK 293F cells for eukaryotic expression, and the supernatant was collected after the 293F cells was cultured.

The same method was used to prepare the sequence encoding eight repeats (named ATHPV8) and ten repeats (named ATHPV10) as comparative proteins.

Elution: The target protein was purified using a HisTrap HP 5 mL affinity chromatography column (Cytiva) in an elution buffer containing 20 mM Tris (pH 8.0), 150 mM NaCl, and 300 mM imidazole.

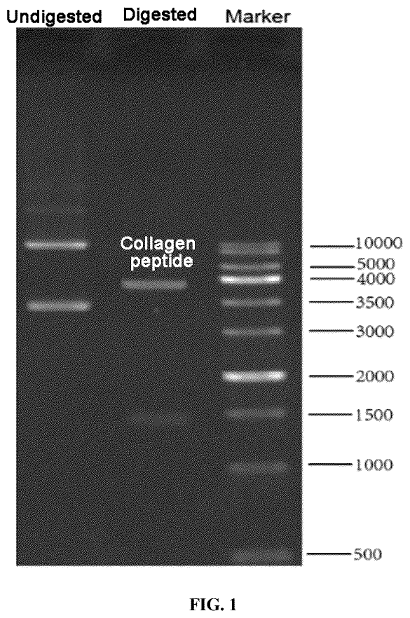

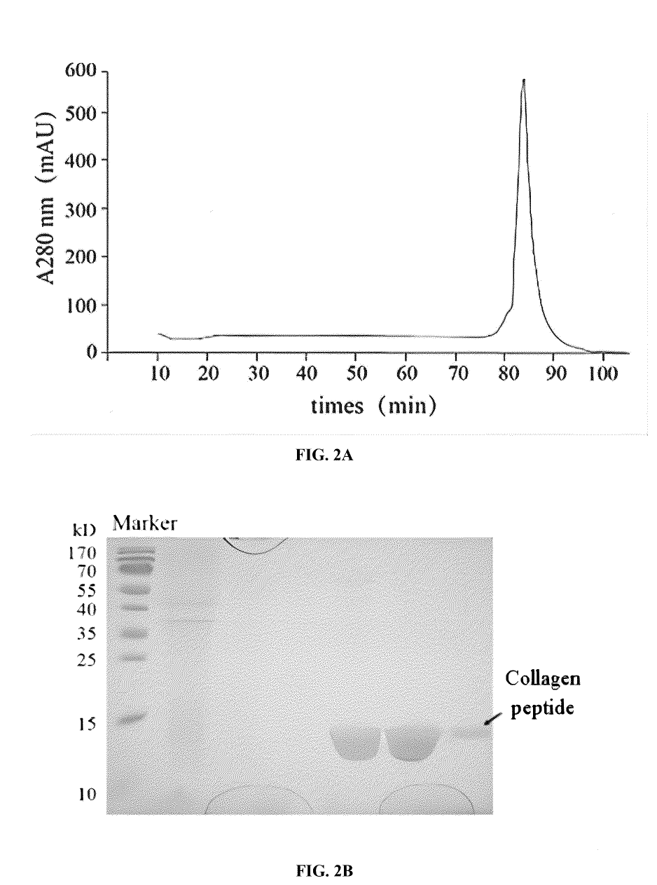

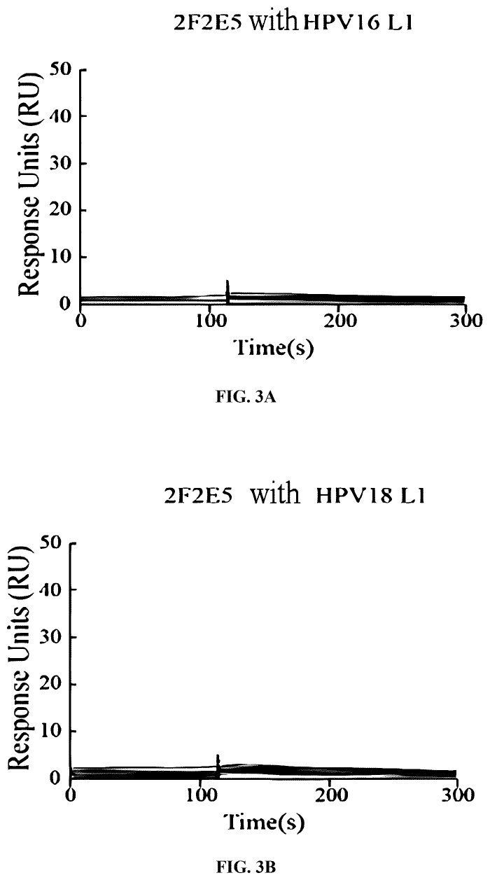

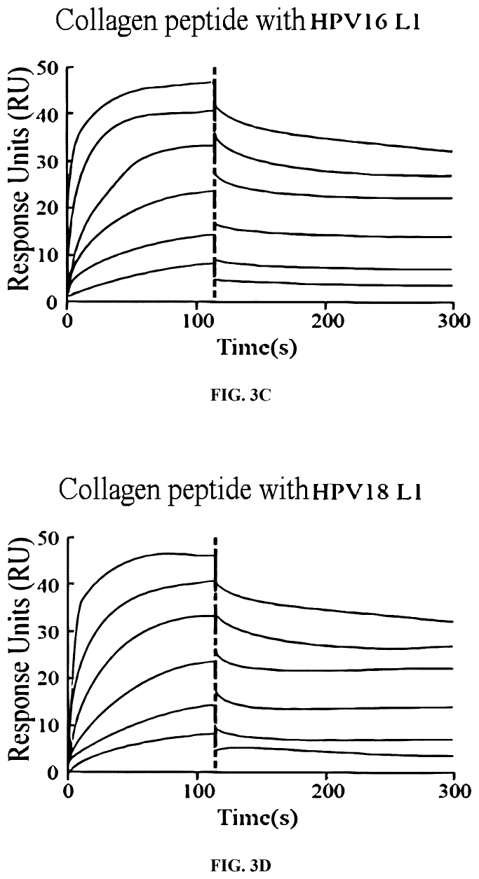

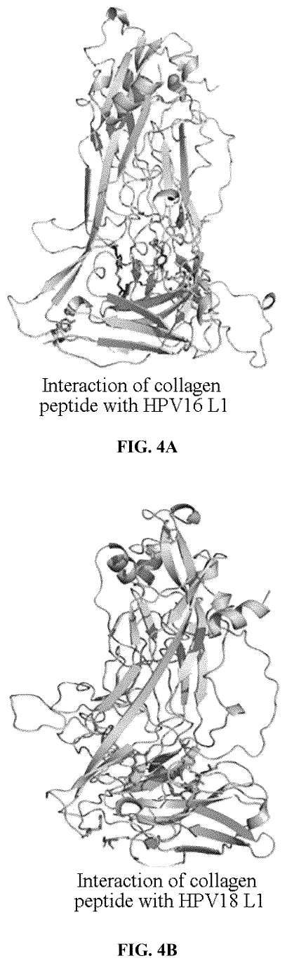

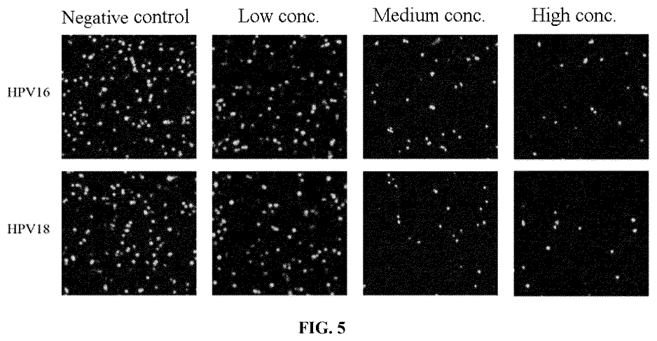

Purification: The protein was further purified using a HiLoad 16/600 Superdex 75 prep gel filtration chromatography column (Cytiva) in a buffer containing 20 mM Tris (pH 8.0) and 150 mM NaCl, at a flow rate of 1 ml/min. The purified protein was identified by SDS-PAGE, with a molecular weight of approximately 13 kD, as shown in FIG. 1 and FIGS. 2A-2B, and was stored at −80° C. for later use.

II. Detection of the Binding Affinity of the Recombinant Type III Human-Derived Collagen Peptide to HPV 16/18 L1 Proteins by SPR Method: HPV 16/18 L1 proteins were immobilized on a Chip CM5 (Cytiva), and the binding kinetics of the fusion protein to HPV 16/18 L1 were detected using a Biacore T200 surface plasmon resonance instrument. The buffer system was Phosphate-Buffered Saline with Tween® 20 (PBST) (10 mM Na₂HPO₄, 2 mM KH₂PO₄, pH 7.4, 137 mM NaCl, 2.7 mM KCl, 0.005% Tween 20).

The diluted ATHPV3, ATHPV8, and ATHPV10 (39, 78, 156, 312.5, 625, 1250 nM) were passed over the chip at a flow rate of 30 μL/min for 2 minutes, followed by a 4-minute dissociation period to detect the binding status with HPV 16/18 L1. After each detection, 10 mM glycine (pH 3.0) was used for regeneration for 2 minutes.

The binding data was fitted using the Langmuir binding equation via Biacore™ T200 evaluation software, with results shown in FIGS. 3A-3D. The results indicated that ATHPV3 could bind to HPV 16/18 L1 with high affinity, with affinities of 51.6 nM and 58.2 nM, respectively, while ATHPV8 and ATHPV10 did not bind to HPV 16/18 L1 proteins, likely due to their larger molecular weights and more complex structures, which prevent them from reaching the binding sites of HPV 16/18 L1.

III. Molecular Docking: Based on the amino acid sequence, the three-dimensional structure of ATHPV3 was predicted using AlphaFold2, with a predicted local distance difference test (pLDDT) average value of 93.562 used to classify the models, selecting the highest-ranked optimized structure. The protein's pLDDT was rendered using UCSF Chimera 1.16. Molecular docking of HPV 16/18 L1 with ATHPV3 was performed using ZDock 3.0.2 software. The results shown in FIGS. 4A-4B indicate that the recombinant type III human-derived collagen peptide ATHPV3 forms five hydrogen bonds with HPV16 L1 and four hydrogen bonds with HPV18 L1, allowing for tight binding between ATHPV3 and both proteins. From the figure, it could be seen that ATHPV3 could enter the groove region of HPV 16/18 L1 for binding. We speculate that the larger molecular weights of ATHPV8 and ATHPV10, due to their more complex conformations, prevent them from inserting into this region, leading to an inability to bind to HPV 16/18 L1 and exert their function.

IV. Detection of the Neutralizing Activity of the Recombinant Type III Human-Derived Collagen Peptide against HPV 16/18 Pseudoviruses by Luciferase Reporter Gene Assay: 293 FT cells were plated at a density of 1×10⁵/mL in a 96-well plate, and HPV 16/18 pseudoviruses were diluted in DMEM culture medium at ratios of 1×10⁻¹, 1×10⁻², 1×10⁻³, 1×10⁻⁴, and 1×10⁻⁵, with 100 μL added to the cells and incubated at 37° C. for 72 hours. Positive wells were identified as those with one or more cytopathic effects observed under an inverted fluorescence microscope. The Reed-Muench method was used to calculate the tissue culture infectious dose 50 (TCID50), which indicates the virus dilution at which half of the cells were infected, with the TCID50 endpoint dilution ratio being 1:1,000.

293 FT cells were plated at a density of 1×10⁵/mL in a 6-well plate, and the structural gene expression plasmid and reporter plasmid were co-transfected into the 293 FT cells using Lipofectamine 2000. The transfected 293 FT-HPV 16/18 L1 cells were plated at a density of 1×10⁴/mL in a 96-well plate, and ATHPV3 was serially diluted in Dulbecco's Modified Eagle Medium (DMEM) containing 10% fetal bovine serum, ranging from 1 μM to 0.05 nM. The diluted recombinant ATHPV3 was then added to the pseudovirus (at a dilution ratio of 1:1,000) in the 96-well plate, with the ATHPV8 group serving as a negative control, and incubated at 37° C. for 1 hour. Afterward, 100 μL of the resulting mixture was added to the 293 FT cell wells and incubated at 37° C. for 24 hours, after which green fluorescence (indicating reporter gene expression) was observed under a fluorescence microscope. The results are shown in FIG. 5, indicating that both high concentration (1 μM) and medium concentration (0.5 μM) ATHPV3 effectively inhibited pseudovirus infection of 293 FT cells, while 1 μM ATHPV8 showed no significant inhibitory effect. The results of the pseudovirus-luciferase reporter gene assay further demonstrate that ATHPV3 neutralizes HPV 16/18 pseudoviruses by binding to HPV 16/18 L1 proteins, effectively reducing viral load.

Finally, it should be noted that the above examples are only intended to illustrate the technical solutions of the present disclosure and do not limit it. Although the present disclosure has been described in detail with reference to the aforementioned examples, those skilled in the art should understand that modifications can still be made to the technical solutions described in the aforementioned examples, or some or all technical features can be equivalently replaced. Such modifications or replacements do not cause the corresponding technical solutions to fall outside the scope of the technical solutions of the present disclosure.

What is claimed is:

1. A recombinant type III human-derived collagen peptide for anti-HPV infection designated as ATHPV3, having the amino acid sequence of SEQ ID NO: 1, wherein the amino acid sequence of SEQ ID NO: 1 comprises three repeats of the sequence corresponding to positions 991 to 1032 of a type III human-derived collagen encoded by UniProtKB: P02461.

2. A gene encoding the recombinant type III human-derived collagen peptide for anti-HPV infection of claim 1, having the nucleotide sequence of SEQ ID NO. 2.

3. A method for preparing the recombinant type III human-derived collagen peptide for anti-HPV infection of claim 1, the method comprising the following steps:
Cultivation: Triplicating the amino acid sequence from positions 991 to 1032 of a type III human-derived collagen encoded by UniProtKB: P02461 and named ATHPV3; ligating a 3′ end of the amino acid sequence to a histidine tag and a termination codon (TGA), and constructing a resulting sequence into a plasmid Cytomegalovirus (CMV)-Actin Globin Gateway Shuttle (pCAGGS) vector through restriction enzyme sites EcoRI and XhoI, transfecting a resulting vector into a human embryonic kidney (HEK) 293-F cell for eukaryotic expression, and collecting a supernatant after the HEK 293F cell is cultured;
Elution: Purifying a target protein using a HisTrap HP 5 mL affinity chromatography column, and eluting the purified protein with a buffer containing pH 8.0, 20 mM Tris, 150 mM NaCl, and 300 mM imidazole; and
Purification: Further purifying the eluted protein using a HiLoad 16/600 Superdex 75 prep gel filtration chromatography column in a buffer containing pH 8.0, 20 mM Tris, and 150 mM NaCl, at a flow rate of 1 ml/min; Identifying the further purified protein by sodium dodecyl sulfate-polyacrylamide gel electrophoresis (SDS-PAGE), and determining a protein with a molecular weight of 13 kDa as a target collagen peptide.

4. A method for preventing and/or treating an HPV infection, comprising administering to a subject in need thereof a therapeutically effective amount of the recombinant type III human-derived collagen peptide for anti-HPV infection of claim 1.

5. The method of claim 4, wherein the recombinant type III human-derived collagen peptide ATHPV3 binds to HPV 16/18 L1 proteins, with affinities of 51.6 nM and 58.2 nM, respectively.

6. The method of claim 5, wherein the recombinant type III human-derived collagen peptide ATHPV3 forms five hydrogen bonds with HPV16 L1 and four hydrogen bonds with HPV18 L1.


SEQUENCE LISTING (2 sequences)

SEQ ID NO: 1 - protein, 126 aa, synthetic construct
  source 1..126: note = Recombinant Type III Human-Derived Collagen Peptide
GPIGPPGPRGNRGERGSEGSPGHPGMPGPPGPPGAPGPCCGGGPIGPPGPRGNRGERGSEGSPGHPGMPGPPGPPGAPGPCCGGGPIGPPGPRGNRGERGSEGSPGHPGMPGPPGPPGAPGPCCGG

SEQ ID NO: 2 - DNA, 318 bp, synthetic construct
  source 1..318: mol_type = other DNA; note = Gene encoding the recombinant type III human-derived collagen peptide for anti-HPV infection
ccgggccatccgggcatgccgggcccgccgggcccgccgggcgcgccgggcccgtgctgcggcggcggcccgattggcccgccgggcccgcgcggcaaccgcggcgaacgcggcagcgaaggcagcccgggccatccgggcatgccgggcccgccgggcccgccgggcgcgccgggcccgtgctgcggcggcggcccgattggcccgccgggcccgcgcggcaaccgcggcgaacgcggcagcgaaggcagcccgggccatccgggcatgccgggcccgccgggcccgccgggcgcgccgggcccgtgctgcggcggc